(19) United States
(12) Patent Application Publication
(10) Pub. No.: US 2004/0110172 A1
(43) Pub. Date: Jun. 10, 2004

(54) Biological results evaluation method

(75) Inventors: N. Eric Olson, Seattle, WA (US); Jeff Kozlowski, Seattle, WA (US)

Correspondence Address: LaRiviere, Grubman & Payne, LLP, P.O. Box 3140, Monterey, CA, 93942, US

(73) Assignee: Vizx Labs, LLC, Seattle, WA (US)

(21) Appl. No.: 10/456,945

(22) Filed: Jun. 6, 2003

Related U.S. Application Data
(60) Provisional application No. 60/386,888, filed on Jun. 6, 2002.

(51) Int. Cl. C12Q 1/68; G06F 19/00; G01N 33/48; G01N 33/50; G06F 11/30; G06F 12/14

(52) U.S. Cl. 435/6; 702/20; 713/200

(57) ABSTRACT

Disclosed is a method and related device (803) for analysis of biological information. With more particularity, disclosed is a novel method and device (803) for storing, using and collaboratively sharing the results of life sciences information. The method and device joins remote users (801) with a central information repository (803) to relate biological information (805) to other datasets such as various internet-based public and private human genome registries (807). As a result, the user is provided with a powerful bioinformatics tool with applications in medical diagnostics, pharmaceutical design and individualized medical treatment.

### RELATED APPLICATION

This application claims priority based upon Provisional Patent Application No. 60/386,888 filed on Jun. 6, 2002.

### TECHNICAL FIELD

The disclosed method and related device pertain to the life science field as well as to the related biomedical field.

### BACKGROUND ART

Microarrays are an emergent tool for biological science and diagnostic use in assaying and understanding gene expression data. These devices are created by adapting the methods of microprocessor manufacturing, resulting in microchips that can contain thousands of distinct DNA probes on glass in place of transistors on silicon. With a chip, a tissue sample and a scanner, a technician can get a detailed picture showing which genes are most active and which have been silenced in the sample.

All the chips generally work on the same principle: the glass is coated with a grid of tiny spots many microns in diameter and each spot contains millions of copies of a short sequence of DNA. Each microarray has a designated layout that identifies which DNA sequences are where. To make their snapshot, scientists extract from their sample cells messenger RNA (mRNA). Using enzymes, they make millions of copies of the mRNA molecules, tag them with fluorescent dye and break them up into short fragments. The tagged fragments are washed over the chip and hybridized with the appropriate target location on the microarray. Although there are occasional mismatches, the millions of probes in each spot ensure that it lights up only if complementary mRNA is present. The brighter the spot fluoresces when scanned by a laser, the more mRNA of that kind was in the cell. Microarray technology has been full of promise, but realizing the full potential of microarray data derived from experiments has yet to occur. Managing, analyzing and relating results to diverse external databases on a gene-by-gene basis under presently known methods can be time consuming, inefficient and even overwhelming. These problems are compounded when a researcher attempts to derive meaningful conclusions from microarrays made by different manufacturers. To date, systems of annotating gene information are not interchangeably standardized.

Array manufacturers provide both a unique identifier such as an Accession Id or Image Clone Id, and annotation for each gene represented on a particular array. This annotation usually consists of the gene name. A common source of this type of information is UniGene. Given the unique identifier for a gene it is possible to determine the current UniGene gene name. This information is updated in the UniGene database approximately every 2 months. The name associated with a particular gene may change when UniGene is updated. In addition, many of the genes in UniGene are designated “Unknown EST” indicating that the gene has not been characterized. As these genes are characterized they are assigned a gene name. In addition, a particular sequence may be assigned to a different gene when UniGene is updated. This may be done to correct errors in the original classification of that sequence. Thus, annotation associated with a particular gene on an array may change with time in at least three different ways. First, the preferred name for that gene may change in some way. Second, “Unknown ESTs” may become known genes and third, the particular sequence on the array may be reassigned to a different gene. Therefore, the annotation provided with a particular array may not accurately reflect what is currently known about that gene.

Consequently, several factors interfere with the ability of a microarray user to compare data from two different array platforms. First, many microarray results analysis software packages cannot accommodate data from multiple vendor platforms. For example, comparing GeneChip data with data from spotted cDNA microarrays may not be possible using one piece of software. Secondly, finding the same gene on two different arrays may be difficult and time consuming because of two factors: the annotation associated with each gene on the two different arrays may be different; and the particular accession id or image clone id chosen to represent that gene on the two arrays may be different. Gene names can change over time and unless annotation is updated frequently, the annotation provided with an array can be out of date. In some cases this could result in the same gene having very different annotations on two different arrays that would not be identifiable as being the same gene. Additionally, if different Image Clones representing the same gene were used on the different arrays, matching the two genes by Image Clone ID would not be possible.

As a result, there has been a long felt need for a comprehensive and non-microarray manufacturer specific method for processing biological information generated from comprehensive testing tools such as microarrays.

The present invention discloses a network-based system and device to solve these and other problems. The system and device combines comprehensive data management, analytical and information mining functions to speed medical diagnostics and more comprehensive awareness of metabolic pathways that lead to a more systematic understanding of medical diseases and disorders, based upon the convenience and benefits of world wide web network access.

### DISCLOSURE OF THE INVENTION

We disclose herein a novel method and device for storing, using and collaboratively sharing the results of life sciences information. The method and device can help to better understand gene expression, and relate the information to other datasets such as various internet-based public and private human genome registries. As a result, a user is provided with a powerful bioinformatics tool with applications in medical diagnostics, pharmaceutical design and individualized-medical treatment.

The system and device relies on and builds upon existing biological understanding, bioinformatics methodologies, Web standards and other data management and analysis practices well-known in the art, including internet protocols, database structures and life science Web services such as UniGene and LocusLink. The system and method automates bioinformatic processing at a level accessible to users without dedicated reliance on bioinformatic specialists and learning of complicated programming techniques. Previous systems have been unduly complicated and require dedicated personnel to carry out even the most routine results analyses. Based upon web browser level of simplicity and quick-response minimal click navigation, the system and device provide a number of unique analytic and other features as it creates a new level of usability and bioinformatics system integration.

Designed for a World Wide Web (web) based platform and configurable for an intranet or internal network, the system and device uses secure network access to password-protected accounts, linked to a password protected relational database with authentication potentially over an HTTPS secure connection. As a direct and intended consequence of web access, the method and device is platform independent, allows for multi-user remote collaboration and requires no special user equipment. Standard computer systems capable of Internet access such as Windows, Linux and Macintosh are representative user devices, but by no means the only ones. Thin client devices are equally capable of accessing the system.

To begin system use, once the user has been authenticated, biological information can be uploaded for individual or collaborative analysis. After biological information has been uploaded, a variety of functions can be performed based upon the type of information. Uploaded biological information can be searched, compared and clustered by function. The searchable database of genes allows the user or users to find and view expression information for specific genes on the input device such a microarray. Genes can then be searched by accession ID, image clone ID or cluster ID. In addition, the pattern navigation tool allows users to also search for genes matching user-defined expression patterns.

Once uploaded, biological information can then undergo a variety of analyses both individually as well as in group use. These analyses start with characteristics provided by the user or users, but can easily include updated information from a variety of sources. One example of biological information query using the disclosed system and device is to determine which genes in the genetic information are differentially expressed. The system and device offers from a variety of normalization and statistical methods, pair-wise and multiple condition comparisons. The results can be used to generate lists and publication-quality graphs for each comparison, with comprehensive, flexible quality control, and gene summaries created for all genes.

In addition to searching options, pair-wise comparisons can be undertaken that include user defined parameters including normalization, statistics and threshold values. Multi group projects allow for comparisons across multiple groups, such as time course studies. Statistical analysis of multi-group projects can also be undertaken using analysis of variants (ANOVA), and biological information can be reviewed more efficiently by using gene based navigation. With more time consuming queries, user feedback such as percent-completion bars for longer analytic functions is provided.

While biological information searching and comparison can be local, the system and device realizes its full potential by providing the user with the latest genetic information via the World Wide Web.

Clustering genes by function using Gene Ontologies enables the user to track biological processes and specific regulatory pathways such as apoptosis at the click of a button. Color coded expression profiling and unique visualization tools make it easy to identify patterns. Web-integrated ‘cluster genes by function’ feature automatically uses latest Gene Ontologies™.

Biological information specific characteristics of the method and device include an integrated information management system that is centered around a relational database to manage and track experiment, target, array and experimental condition information. Biological information can then be organized by input device such as array, or by condition. Unlike many proprietary systems, the disclosed method and device will accept biological information in multiple formats including Affymetrix, Pathways and Scanalyze. It is also easily modified to allow additional formats, including custom user defined biological information formats and stores cDNA target information and experiment annotation in addition to raw data.

Quality control of biological information can be undertaken to screen for input device errors such as poor spot quality or low intensity values which are accounted for with automated quality control mechanisms or can be addressed with user-defined parameters. The data management system tracks experiment, target, array, experimental condition and annotation information. User efforts are consequently optimized through the screening and removal of undesired low quality data.

After the biological information has been screened for quality, it is presented to the user. To do so, graphical expression profile summary screens employ color-coded data visualization for up/down regulation. Scatter plot can be used for visualization of pairwise comparisons and the interactive design allows for rapid identification of differentially expressed genes with direct access to raw data and gene information. Publication quality graphs including standard error bars generated for all analyses can be returned to users on demand.

One advantage of the present system and device is based upon the fact that biological information interpretation takes place with more current updates than stand alone systems can provide. By drawing from automatic biological information summaries created from web based data sources such as UniGene and LocusLink, plus click-throughs to other databases such as Homologene, Genbank, GeneCards and OMIM, the user is able to take advantage of up to date biological information when generating results. The user can also retrieve sequences and store the retrieved sequences as part of the annotation for genes on input devices such as arrays. Another benefit of the present system and device is that previously unknown biological information such as an unknown EST is automatically updated when known. The most current UniGene information is automatically integrated and displayed for each gene corresponding to a particular input device such as a microarray. As a consequence, the most current genetic information available through public databases is displayed based upon automatic integration of current UniGene and LocusLink information for each gene on a device such as a microarray. Links to external databases such as GenBank, UniGene, Homologene, OMIM, LocusLink, and GeneCards™ broaden the possible coverage of genetic information. Finally, functionality of Integrated Blast and Primer design is available for retrieved genetic sequences.

During system and device operation, there are 5 main types of user defined data according to the disclosed system: Arrays, Conditions, Targets, Experiments and Projects.

Arrays refer to microarrays. These are substrates with anywhere from a few to tens of thousands of genes on them. Analyzer stores annotation about each gene on a given array. Arrays can be either purchased commercially or custom made in the laboratory.

Conditions can be thought of as general groupings. For example, in a cancer study a user might have one set of patients without cancer and one set of patients with cancer. Patients without cancer would be grouped under a condition called ‘Normal’ whereas patients with cancer would be grouped under a condition called ‘Cancer.’

Targets refer to a cDNA/mRNA sample. In the cancer study, the user might take a cDNA sample from each patient. By way of definition, a cDNA sample from a cancerous patient would be of the condition ‘Cancer’ and a sample from a non-cancerous patient would be of the condition ‘Normal.’

An Experiment refers to the combination of a Target and a data source such as an Array. With greater particularity, the user or assistant to the user exposes cDNA to an array and receives a set results. For example, cDNA from patient 5 (condition ‘Cancer’) is exposed to Array U95A.

A Project is a set of experiments. In a project experiments of similar conditions are grouped together. The combined results are then compared to other groups. In the cancer example, the experiments from the normal patients are combined and the experiments from the cancerous patients are combined. Now, one can look for differences between the two groups. A project can contain any number of groups, but must have at least two.

Enhancements and extensions to the system are possible and many should be apparent to a practitioner of normal skill in the art. Though the disclosure addresses a web-based system shared by many biologists as a preferred embodiment, many of its aspects could be functionally realized in other forms as well, such as a standard operating system application, closed network based system, embedded system on a dedicated or palm type device, or even specialized electronic hardware.

### BRIEF DESCRIPTION OF THE DRAWINGS

FIG. 1a is a screen shot of the entry point of the system.

FIG. 1b is a screen shot of the Upload Wizard initial page.

FIG. 1c is a screen shot of the Upload Wizard to select target.

FIG. 1d is a screen shot of the Upload Wizard to create new target.

FIG. 1e is a screen shot of the Upload Wizard to select data file.

FIG. 1f is a screen shot of the Upload Wizard to save data.

FIG. 1g is a screen shot of the Upload Wizard confirming data saved.

FIG. 2a is a screen shot of the Inventories experiments list.

FIG. 2b is a screen shot of the Inventories experiments detail page.

FIG. 3a is a screen shot of the Pairwise Comparison section to select array.

FIG. 3b is a screen shot of the Pairwise Comparison section for set up comparison.

FIG. 3c is a screen shot of the Pairwise Comparison section for gene list.

FIG. 3d is a screen shot of the Pairwise Comparison section for gene summary.

FIG. 3e is a screen shot of the Pairwise Comparison section for scatterplot.

FIG. 3f is a screen shot of the Pairwise Comparison section to export results.

FIG. 4a is a screen shot of the Project Analysis section for project selection.

FIG. 4b is a screen shot of the Project Analysis section for gene navigation.

FIG. 4c is a screen shot of the Project Analysis section for expression summaries.

FIG. 4d is a screen shot of the Project Analysis section for gene summary.

FIG. 4e is a screen shot of the Project Analysis section for pattern navigation.

FIG. 4f is a screen shot of the Project Analysis section for pattern summaries.

FIG. 5 is a screen shot of the User Preferences section of the system.

FIG. 6a is a screen shot of the Create New Project section for array selection.

FIG. 6b is a screen shot of the Create New Project section for condition selection.

FIG. 6c is a screen shot of the Create New Project section for experiment selection.

FIG. 6d is a screen shot of the Create New Project section with the project created.

FIG. 7 is a flowchart of the method.

FIG. 8 is a system schematic.

FIG. 9 is a screen capture of the Gene Ontologies portion of the method.

FIG. 10 is a relational database schematic.

FIG. 11 is a system overview.

### BEST MODES FOR CARRYING OUT THE INVENTION

Please note, identical articles will be identified with the same number designation throughout the figures.

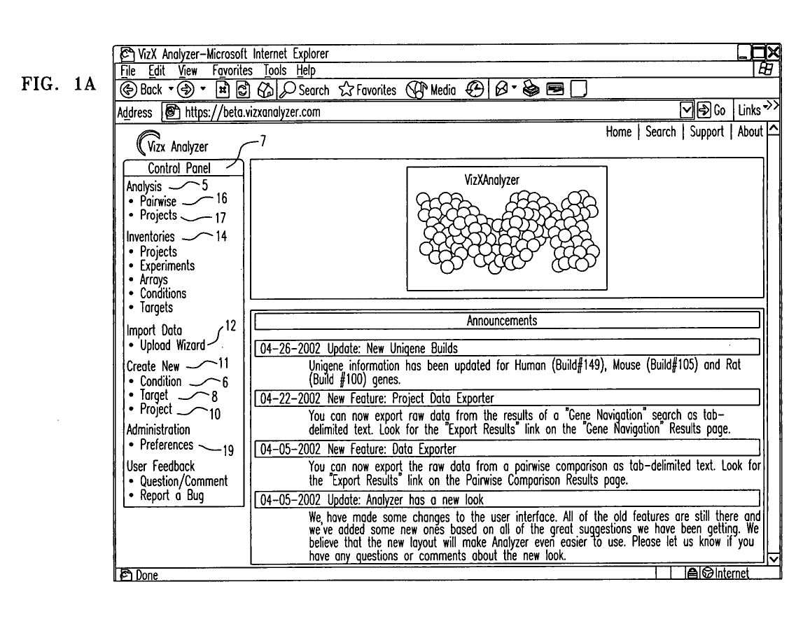

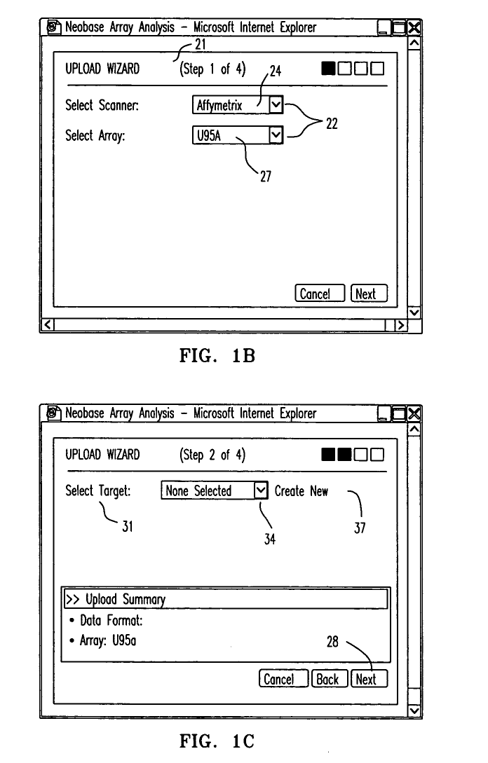

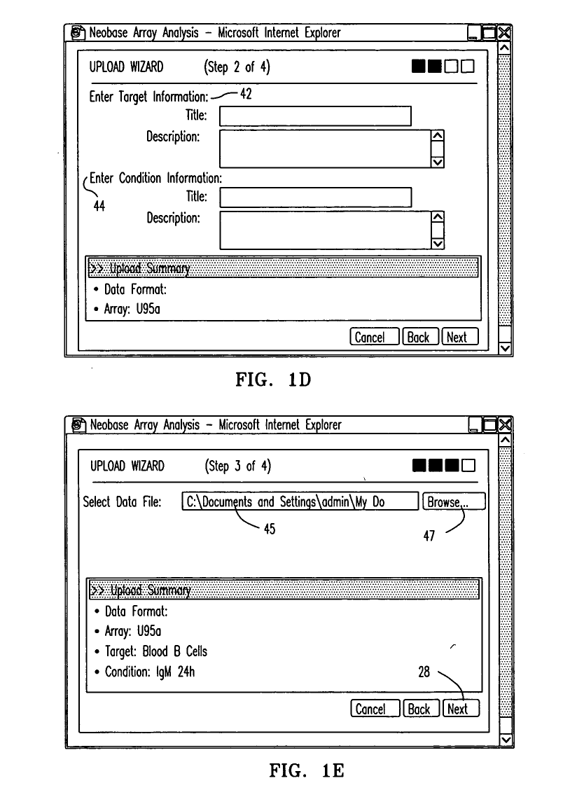

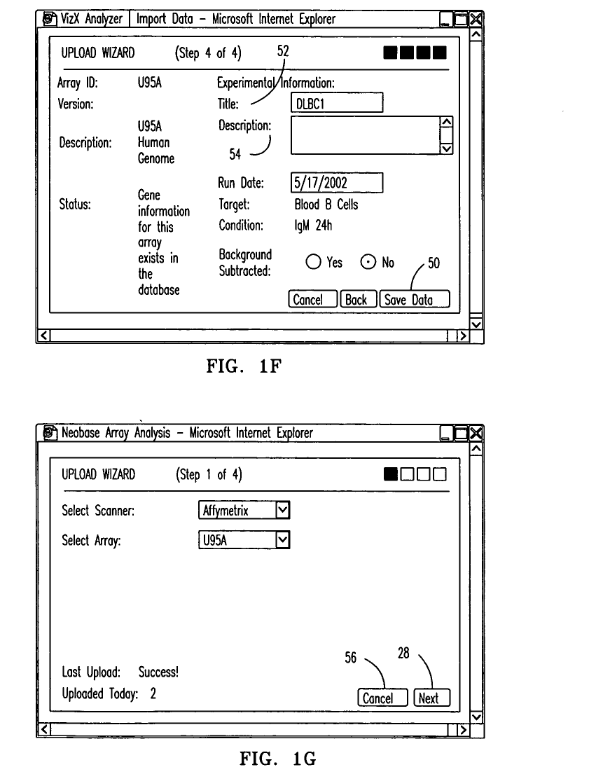

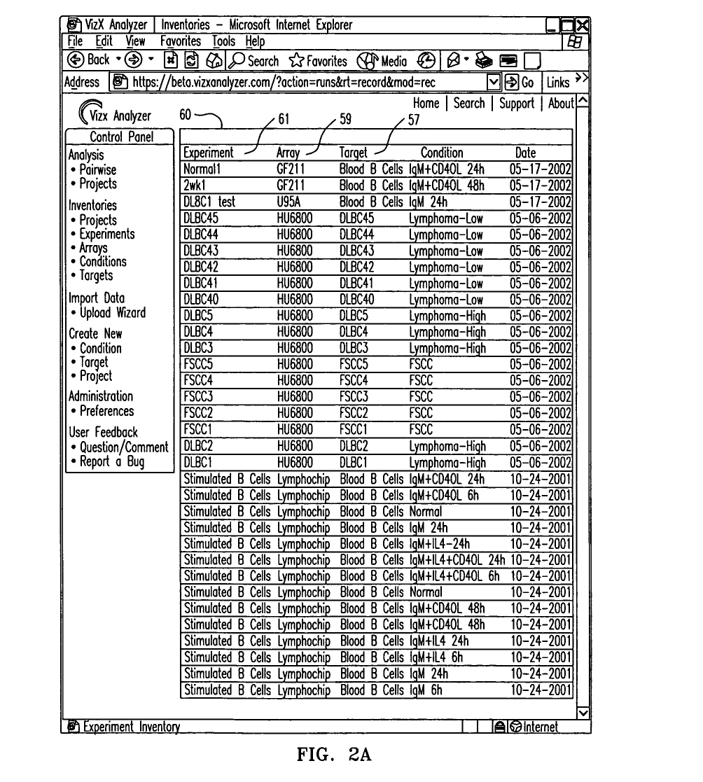

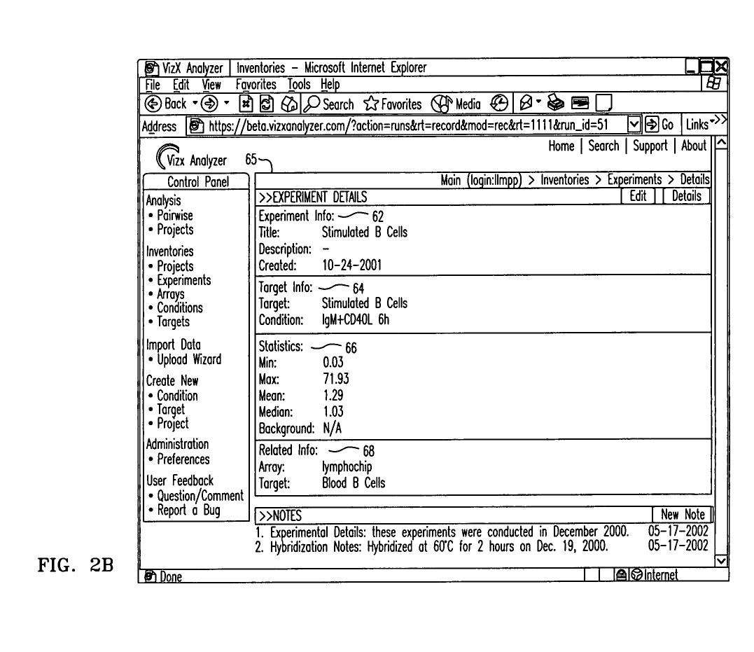

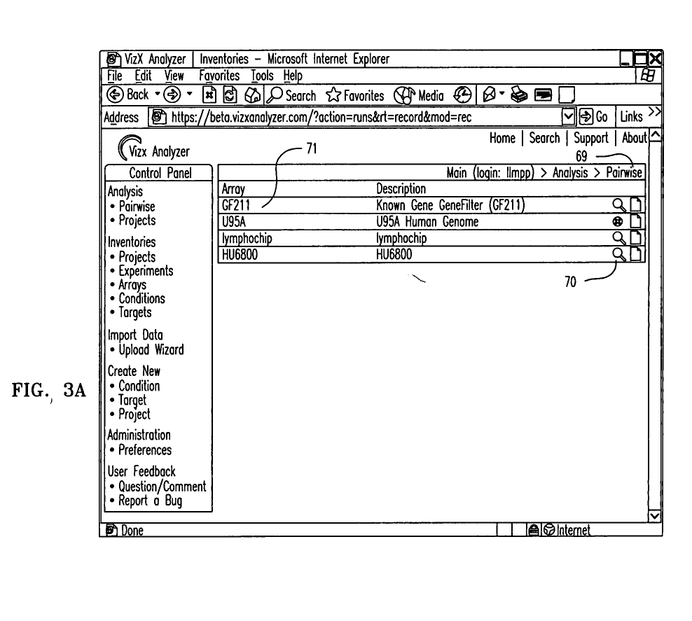

### FIG. 1A Home

FIG. 1A depicts the entry point for the users of the method and related device. The user accesses primary functionality through the use of the Control panel 7 to navigate to other functional screens. To upload data, the user selects the Upload wizard 12 from Control panel 7 on the left. Inventories 14 provides for display of uploaded information. Data analysis takes place through either the Pairwise entry 16 or the Projects entry 17. To perform Pairwise analysis, the user selects Pairwise 16 under the Analysis menu 5 from Control panel 7. For project analysis, the user selects Projects 17 from Analysis menu 5 in Control panel 7. User specific characteristics are set using the Preferences 19 which provides control over the format and structure by which information is displayed. For defining particular information sets, the Create new 11 selection allows for the creation of a new Condition 6, new Target 8 and new Project 10.

### FIG. 1B Upload Wizard 21

To import data according to the method and related device the user can use the Upload wizard 21. Array platform and layouts are selected from the pull down menus 22. The user can select array platform, or software used for image analysis. The user can select the Image analysis software used to generate a raw data file (e.g. Spot On) from the pull-down menu 24. A new pull down menu with available array formats will appear. Select the array format and then click the Next button 28 or select Array layout 27 from list of available options.

### FIG. 1C Upload Wizard Select Target 31

The user can now select the cDNA target used for channel 1. If the target used is not available in the list 34 of available targets 31, the user can select the Create new button 37 to enter information for that target. Select Next 28 when all the needed information has been entered. Note on conditions: The user-defined conditions will be used to group experiments. The user should use the same condition label for each member of a set of replicates. If an array has more than one channel, repeat the steps for Create new 37 for the additional channel or channels.

### FIG. 1D Upload Wizard Create New Target

If a new target is created, the user will need to enter Target information 42 and select an appropriate experimental Condition 44 from the pull down menu. Once complete, the user can create a new condition 44 if desired condition is not in the list of available conditions. The user creates a new condition by selecting Create new 37.

### FIG. 1E Upload Wizard Select Data File

To upload data from a local computer drive or networked data source, the user selects the Browse button 47 to find a data file 45 on a local computer (not shown) or networked device (not shown). The file is selected 45 through the data path window and data upload begins once the user clicks Next button 28. This action will upload the file to the central data repository (not shown).

### FIG. 1F Upload Wizard Save Data 50

The user will now see a summary of the information provided, and can edit the Title 52 and enter a Description 54 for the experiment(s) being uploaded. Selecting the Save data button 50 will save the data. With respect to ‘Spot On:’ the system saves the intensity data for each channel as a separate experiment. The experiments will have the same default title, with channel 1 or channel 2 appended to the title.

### FIG. 1 G Upload Wizard Data Saved

The user can then either upload more data by selecting Next 28 or exit the Upload Wizard by selecting Cancel 56.

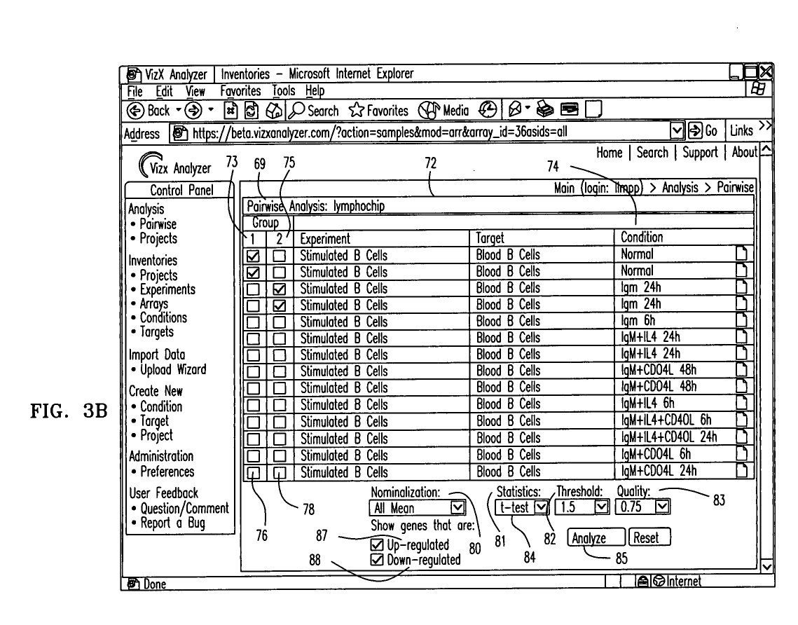

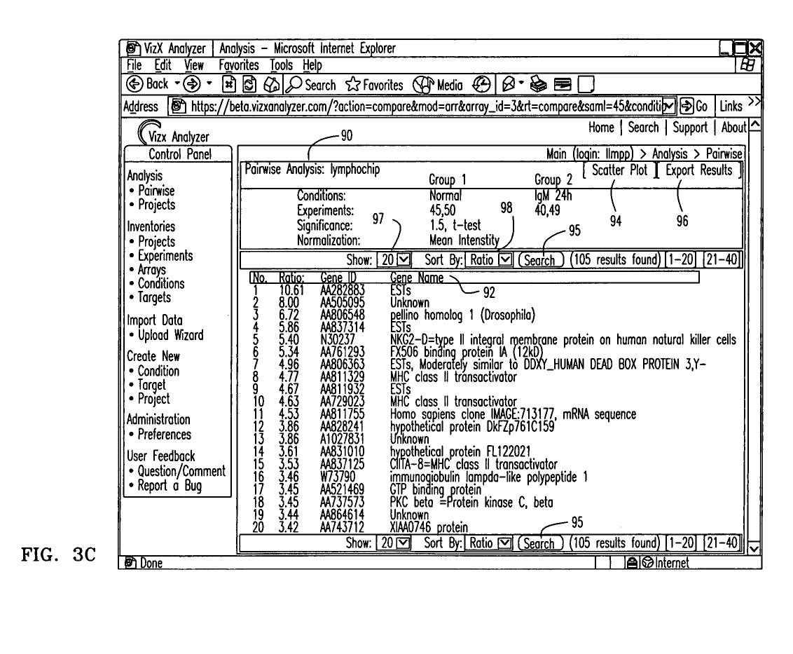

### FIG. 2A Inventories Experiments List 60

An Experiment 61 refers to the combination of a Target 57 and a data source such as an Array 59. For example, the user can expose cDNA (not shown) to Array 61 and receive a set results. With even more particularity, Experiment 61 could take the form of cDNA from a patient (not shown) which is then exposed to Array U95A (not shown). A major functional benefit of the method and related device pertains to the retention of previous experiments and their subsequent accessibility by the user and by invited guests of the user for collaborative purposes. Experiments 61 can be selected from Experiments List 60 and retrieved. Displayed results of Experiments List 60 can be saved as text and also be used in other applications such as Excel™ (not shown).

### FIG. 2B Inventories Experiments Detail Page

Once a particular Experiment 61 is selected, Experiment Detail 65 is displayed. Experiment Detail 65 includes Experiment title, description and creation information 62. Target information includes target name and condition 64. Experiment details also include statistical information 66 and related array and target information 68.

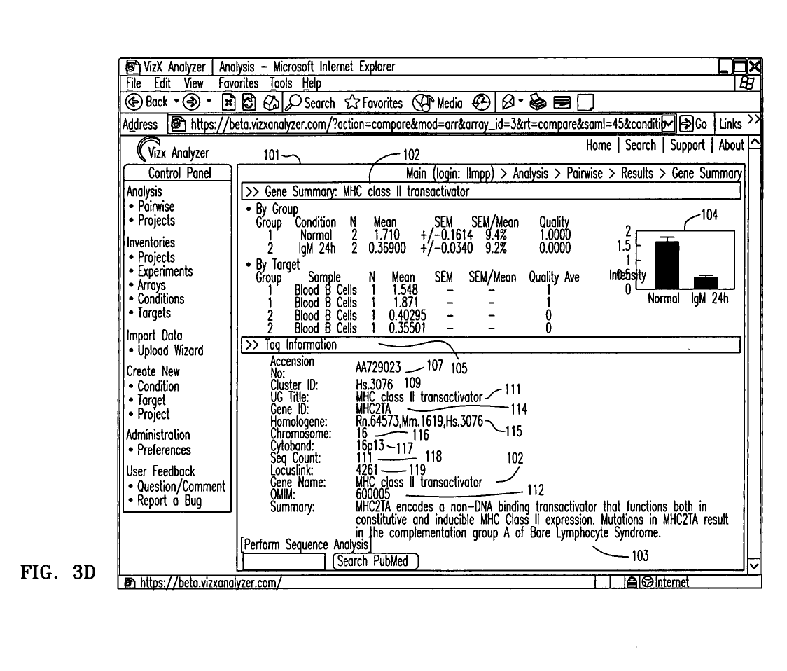

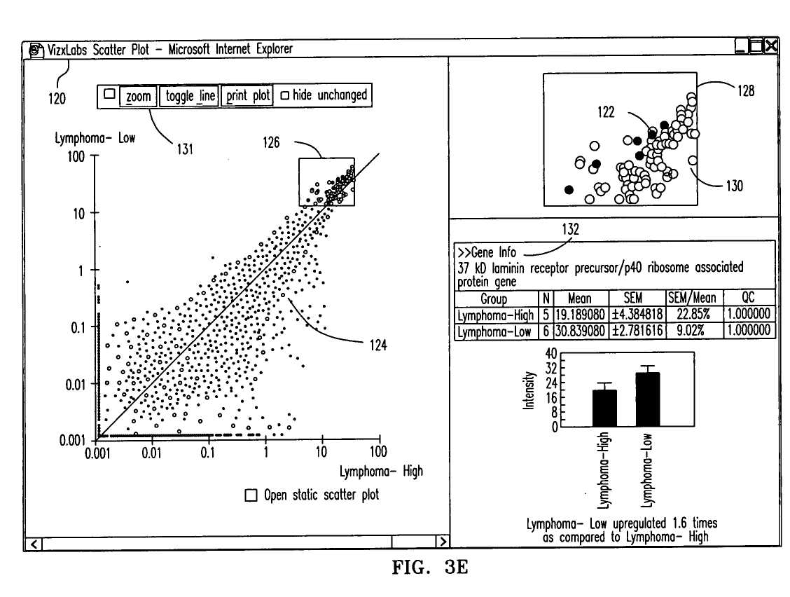

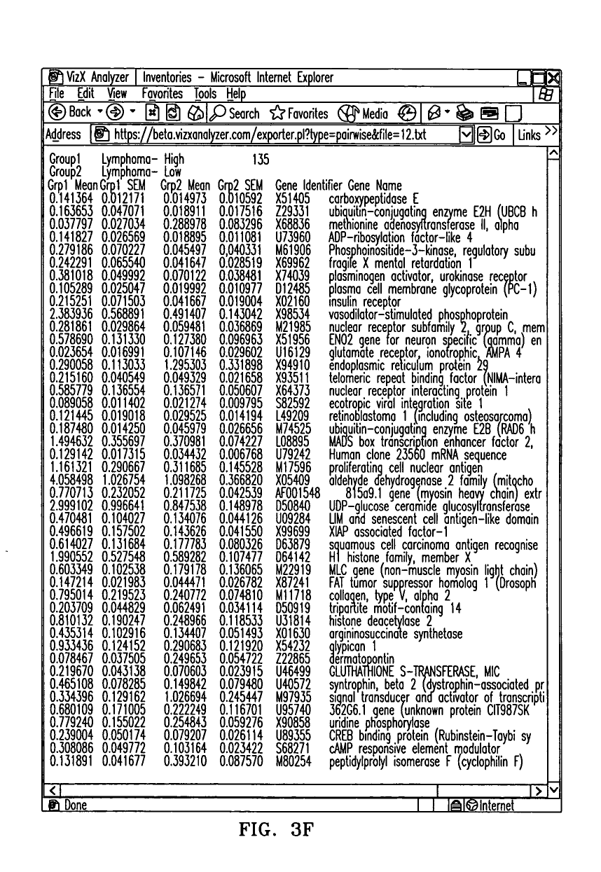

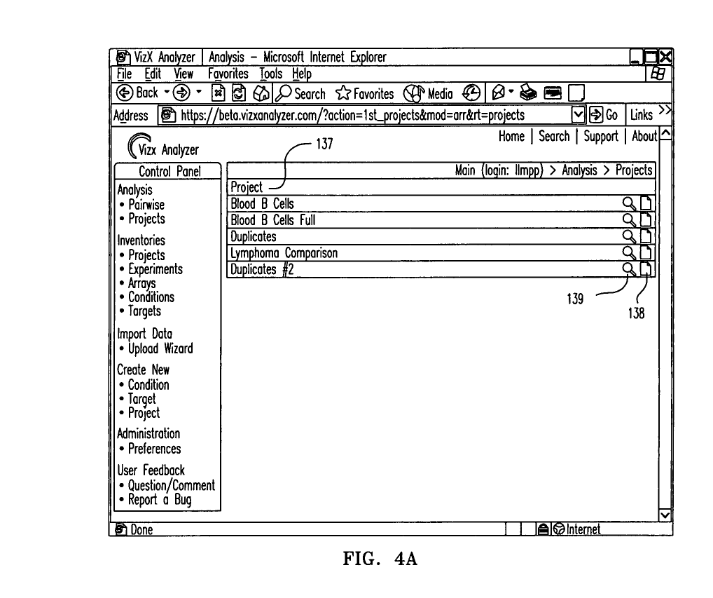

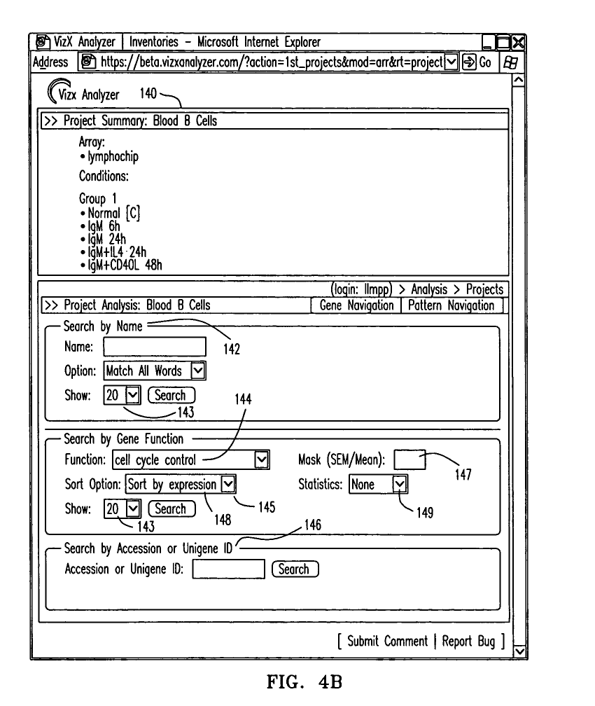

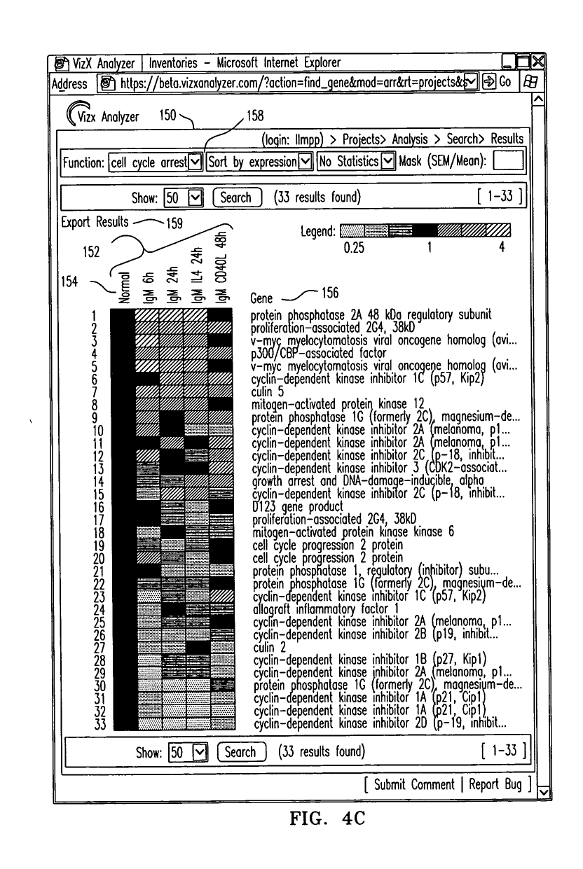

### FIG. 3A Pairwise Comparison Select Array

Pairwise comparison 69 allows the user to set up two groups of data and look for genes that are differentially expressed in two different conditions. If the user has uploaded data there will be a list of available array formats. The user begins by selecting the Analyze Icon (a magnifying glass) 70 to set up a pairwise comparison for a particular array 71.

### FIG. 3B Pair Wise Comparison Set Up Comparison

All available experiments performed using the selected array 72 are listed. The experiments are grouped by condition 74. The user selects the experiments to use for Group One 73 by selecting the boxes in the Group One column 76 for those experiments 72. The user then select the experiments to use for Group Two 75 by selecting the boxes in the Group Two column 78 for those experiments 72. The data from all the experiments will be averaged after normalization. This is achieved by the selection of a Normalization method 80, Statistical test 81, Threshold 82 and Quality control 83 for the comparison. The user selects a normalization method from the Normalization pull-down menu 80. “HKG Mean” (not shown) may not be available for all arrays. The user then selects a method for determining significance from Statistics pull-down menu 81. Selecting t-test 84 will return only genes where the p-value for the difference is less than 0.05. After Statistics 81, the user selects a threshold from the Threshold pull-down menu 82. This number sets the threshold for up or down regulation in group 2 relative to group 1 (e.g., Setting to 1.5 would select only genes that are differentially expressed by at least a factor of 1.5 in group 2 relative to group 1). Finally, the user can select a Quality control cut-off value 83 for the data. This value 83 is calculated differently for different image analysis software (not shown). For Pathways 2—this value is the intensity divided by background, so setting this value to 1.5 would filter out genes where the intensity is less than 1.5 times background. For Spot-On—this value is the intensity divided by background, so setting this value to 1.5 would filter out genes where the intensity is less than 1.5 times background. For Affymetrix—this reflects the Absolute. Call. Setting to N/A ignores this, setting to 0.5 excludes “A” values, 0.75 also excludes “M” values. Using a setting of 0.75 would insure that only genes that are present are included for analysis. For Lymphochip, this value is generated by the image analysis software, and how good the initial measurement of spot intensity was. Setting to a value of 0.75 would insure that only high quality spots are included for analysis.

To display up-regulation 87 or down-regulation 88 the corresponding boxes can be checked by the user. To perform the comparison, the Analyze button 85 is pressed. After the analysis is performed a list of differentially expressed genes will be displayed.

### FIG. 3C Pair Wise Comparison Gene List 90

After submitting a pairwise comparison, genes which are differentially expressed based on user-defined criteria are listed 90. The genes are ordered such that the genes which are most differentially expressed are at the top of the list. The colored arrow indicates whether expression is higher (red) or lower (green) in group two compared to group one. To view more information about any gene in the list select the Gene name 92. Additional information about that gene will then be displayed. Text to the right of the Search button 95 will indicate how many genes were identified. Only part of the gene list is displayed at any one time. The default is to display twenty genes at a time. To display more on each page increase the number in the Show pull down menu 97 and select Search 95. The user can move to the next page of genes by selecting from the ranges. After performing a pairwise comparison using a t-test the list can be sorted by p-value by selecting p-value from the Sort By pull down menu 98 and then selecting Search button 95. The genes will be sorted such that the genes with the lowest p-value are displayed first. In addition, for graphical representation the user selects the Scatterplot link 94 to view a scatter plot of all data for the comparison.

To conclude, the user may select the Export Results link 96 to export the results of Pairwise comparison 90. This will open a new window containing the results in tab-delimited format. These results can be saved and then viewed in Excel™ or shared with other users.

### FIG. 3D Pair Wise Comparison Gene Summary 101

FIG. 3D details Gene summary information from on-line resources such as UniGene and LocusLink. Gene summary includes Gene name 102 and Statistical information 104. Tag information 105 includes the Accession number 107, the Cluster id 109, the UG title 111, the Gene id 114, the Homologene identifier 115, the Chromosome 116, the Cytoband 117, the Sequence count 118, the LocusLink identifier 119, the Gene name 102, the OMIM number 112 and the Summary 103. By selecting the links in gene info the system and device connects to external databases (not shown) such as Genbank, OMIM, GeneCards and others.

### FIG. 3E Pair Wise Comparison Scatter Plot 120

After performing a pairwise comparison the data can be viewed as a Scatterplot 120 with the log intensities for group 1 plotted against the log intensities for group 2. From the Pairwise comparison results page the user selects Scatterplot to view the scatterplot for that comparison. This plot displays the data for all of the genes and color codes the differentially expressed genes. Red points 122 are genes that are expressed at significantly higher levels in group 2. Green points 124 are genes that are expressed at significantly lower levels in Group Two. Gray points represent genes that are not differentially expressed based on the criteria selected for the pairwise comparison. The user then drags the blue box 126 over a region of interest on the graph, and the user can identify spots by mousing over them in the Zoom box. By selecting Zoom 131 the region will be magnified in the Box on the upper right 128. Moving the mouse over a data point 130 will display the name above the box. Clicking on a spot will bring up the Summary information 132 about that spot and associated gene in the lower right panel.

### FIG. 3F Pair Wise Comparison Export Results

The Displayed results 135 can be saved as text and then used in other applications such as Excel™. As a direct and intended function of the method and related device structure, Displayed results 135 can also be viewed by multiple users at the same time for collaborative purposes.

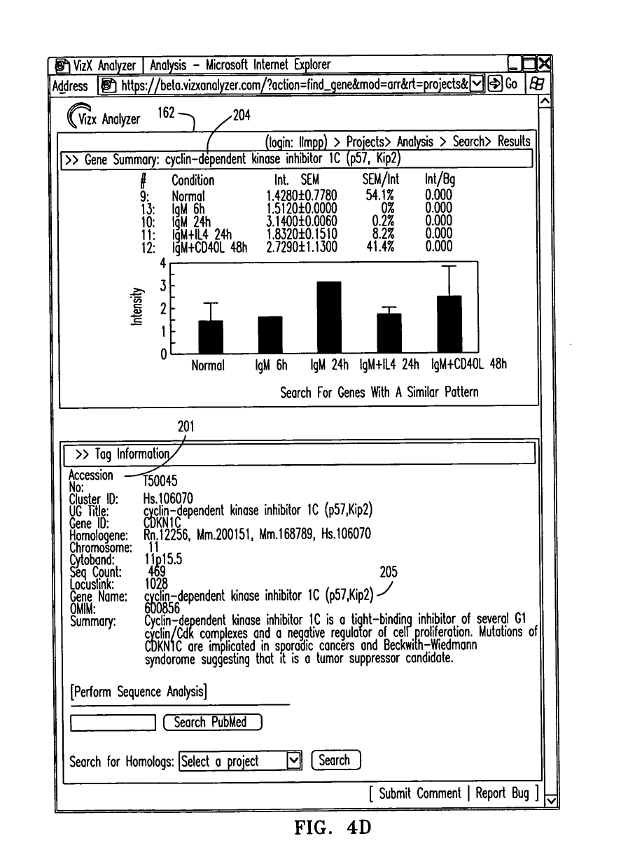

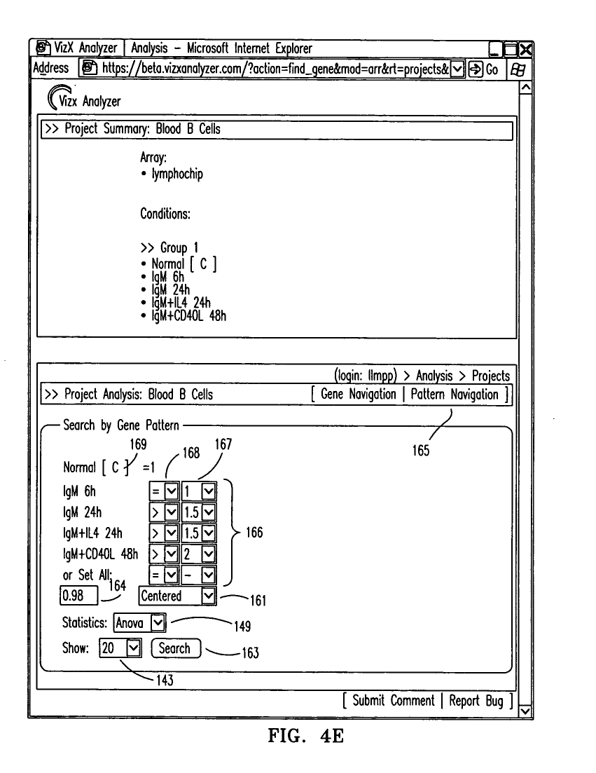

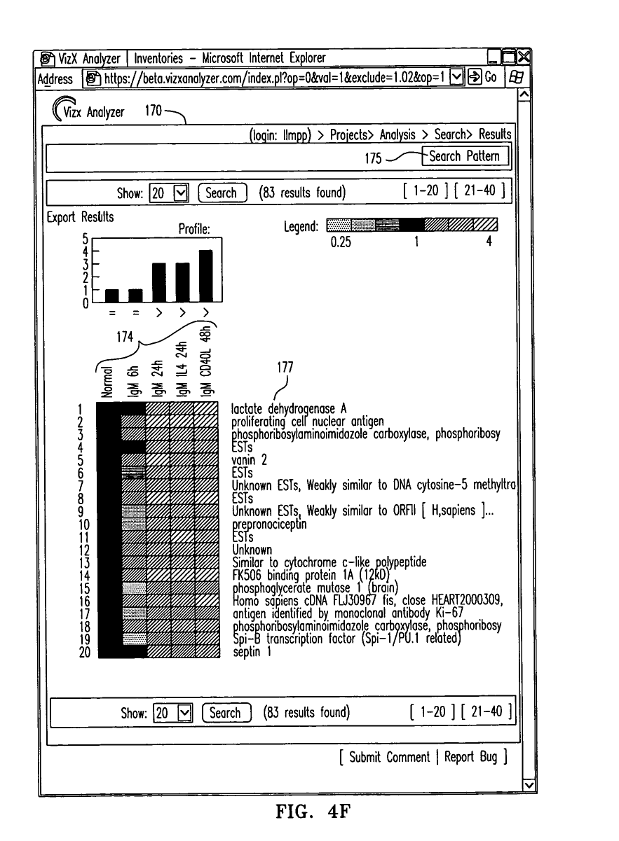

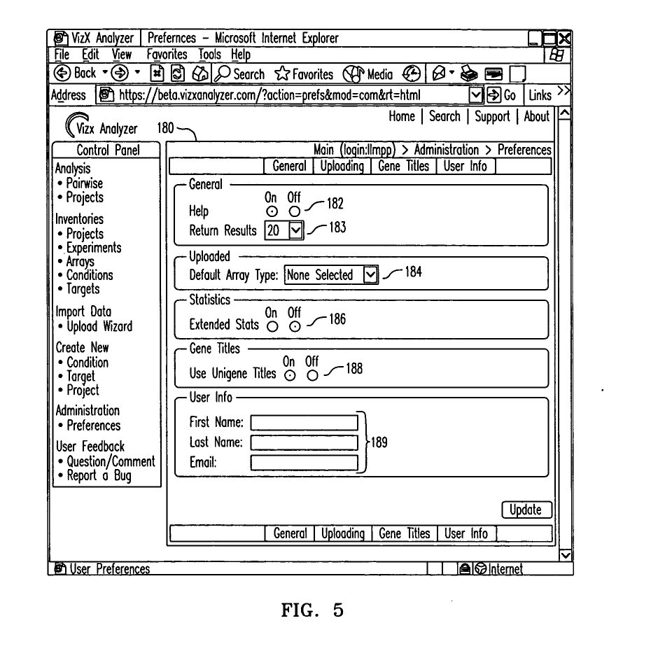

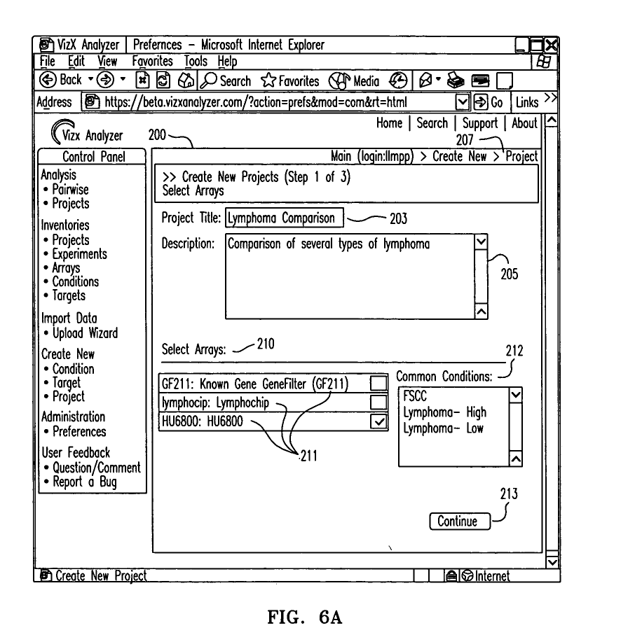

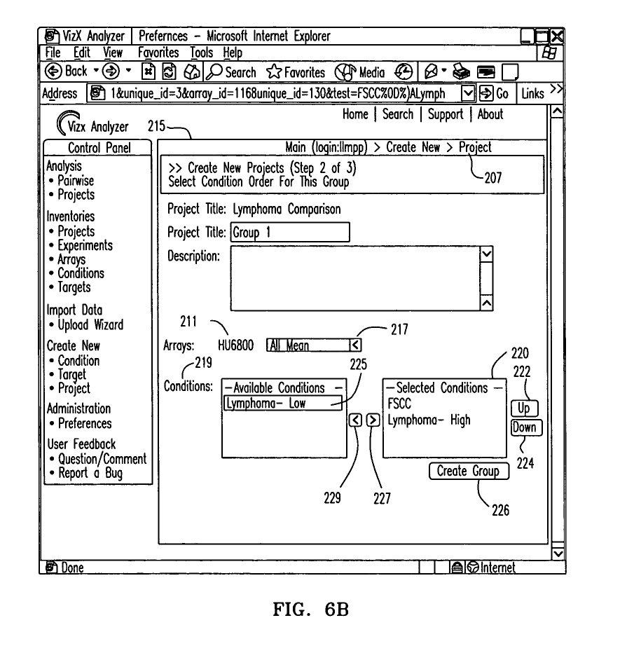

### FIG. 4A Project Analysis Select Project 137

A Project 137 is a user-defined set of experiments. In a project experiments of similar conditions are grouped together. The combined results are then compared to other groups. In the cancer example to follow, the experiments from the normal patients are combined and the experiments from the cancerous patients are combined. As a direct and intended consequence of Project analysis, the user can look for differences between the two groups. A project can contain any number of groups, but must have at least two. To begin, the user selects the Analysis icon 139 for a project in the list. Selection of the Information icon 138 will result in display of information about a project. Next to the Information icon is magnifying glass shaped Analysis icon 139 for the project to be analyzed from the list of available projects.

### FIG. 4B Project Analysis Gene Navigation 140

Clustering genes by Gene Function using Gene Ontologies™. The present system and device provides several features that allow users to view expression profiles of groups of genes selected based on their biological function. The system and device can provide UniGene and LocusLink summary information for each gene on an array. The system and related device integrates Gene Ontology™ designations from LocusLink into this annotation. As new ontology designations are added to LocusLink, this information is automatically added to the annotation for a user's genes. Users can then search for groups of genes on their arrays using this information. Gene navigation allows the user to view expression profile from selected genes for your project. There are three ways that genes may be selected. The first, Search by Name begins with the user entering a Gene name 142. The annotation for the genes contained in the project will be searched for the name entered. The user enters a gene name or part of a Gene name 142 in the text box which is followed by a search of the annotation for genes found on arrays in the selected project. The second searching method, Search by gene function 144 begins with the selection of a biological process ontology from the pull down menu 144. All genes in that project which have that ontology designation will be found.

The Search by gene function 144 method for Project analysis provides a list of available Gene Ontolgies™. An ontology of interest can be selected and a search performed. All genes on the arrays included in that project and having that ontology designation as part of their annotation are selected and an expression profile for each of the genes is created. Gene sets can then be sorted based on expression profile and statistical analysis can be applied to these datasets. These features allow users to view their expression data in the context of biological processes. Search by Accession or UniGene ID 146. User can enter an identifier such as Accession ID or UniGene identifier and search for that particular identifier as well as any additional identifiers that represent the same UniGene cluster. Based upon the Accession Number, the corresponding cluster is found from UniGene. Subsequently, the ID numbers for other sequences of the same cluster can be found and compared to the user's array.

Parameters apply on a context specific basis and include the following options: The Show option 143 controls how many genes will be displayed on page at one time. The Sort option 145 controls how genes are sorted for display. The Sort by expression variant 148 puts genes that are expressed at higher levels than the control at the top of the list and those expressed at lower levels at the bottom. The Mask feature 147 allows the user to mask out intensity values where the SEM is large relative to the mean for a particular expression. Entering 0.25 would gray out conditions where the ratio of SEM to the mean is greater than 0.25. The Statistics option 149 provides for a variety of statistical analyses. Selecting Anova (not shown) will perform analysis of variance for each gene profile to determine whether there are significant differences in expression for that gene across the project. Significance is determined at 0.05 and is indicated by a blue star to the right of the expression profile.

### FIG. 4C Project Analysis Expression Summaries 150

FIG. 4C displays Expression profiles 152 for genes selected. The color-coding indicates changes in gene expression relative to the first group. The user selects the Profile 154 or the Gene name 156 to view more information about the gene. To launch another search or view more genes, the user selects the Control bar 158 at the top. Selection of Export results 159 will export the results of this analysis in a database acceptable data format such as tab delimited format.

### FIG. 4D Project Analysis Gene Summary 162

FIG. 4D depicts Gene summary information 162 from data sources such as UniGene and Locuslink. The user selects the links in gene info to connect to external databases (not shown) such as Genbank, OMIM, GeneCards and others. By connecting to external databases, Gene summary 162 results in the creation of current UniGene and LocusLink summaries for genes.

Array manufacturers provide both a unique identifier such as an Accession Id 201 or Image Clone Id (not shown), and annotation for each gene represented on a particular array. This annotation usually consists of the Gene name 204. A common source of this type of information is UniGene. Given the unique identifier for a gene it is possible to determine the current UniGene gene name 205. At this time, the information is updated in the UniGene database approximately every 2 months. The name associated with a particular gene may change when UniGene is updated. In addition, many of the genes in UniGene are designated “Unknown EST” indicating that the gene has not been characterized. As these genes are characterized they are assigned a gene name. In addition, a particular sequence may be assigned to a different gene when UniGene is updated. This may be done to correct errors in the original classification of that sequence. Thus, annotation associated with a particular gene on an array may change with time in at least three different ways. 1) The preferred name for that gene may change in some way, 2) “Unknown ESTs” may become known genes, and 3) the particular sequence on the array may be reassigned to a different gene. Therefore, the annotation provided with a particular array may not accurately reflect what is currently known about that gene.

The disclosed system and device provides methods for automatically providing the most current information for genes on arrays being analyzed. A representative biological information sample is provided on Table 1. Table 1 shows the increase in gene annotation after an Unknown EST sample is processed according to the present method and related device. Part A shows the annotation provided by array manufacturer. Part B shows the Annotation according to the method and device. At the time of manufacture in 2000 of the array utilized, this gene was designated “Unknown EST”. In October 2001, this gene was characterized and described in UniGene, but the benefit of this additional information would not be as easily available to a user without the present method and related device. To attain the updated information, UniGene and LocusLink summary information is downloaded from the National Center for Biotechnology Information (NCBI) and parsed and stored in a relational database (not shown). The UniGene summary file contains information such as gene title and LocusLink ID for each UniGene cluster. It also contains a list of all Accession Ids 201 and Image Clone IDs that are included in that cluster. Information from LocusLink is also stored in the system and related device associated database. The claimed system and device can then use the Accession Id 201 or Image Clone Id provided by the array manufacturer to look up the current UniGene and LocusLink information for any gene present on an array. When UniGene is updated the new summary information can be incorporated into the system database and this new information will be automatically presented as Gene Summary information for genes on the array, ensuring that users always have the most current UniGene information available.

TABLE 1

| | |
|---|---|
| A. | AA283087 Unknown ESTs |
| B. | Accession No.: AA |
| | Cluster ID: Hs.89104 |
| | UG Title: Homo sapiens BIC noncoding mRNA, complete sequence |
| | Gene ID: BIC |
| | Homologene:- |
| | Chromosome: 21 |
| | Cytoband:- |
| | Seq Count: 24 |
| | Locuslink: 114614 |

### FIG. 4E Project Analysis Pattern Navigation 165

Pattern navigation 165 allows the user to look for genes whose expression profile matches a User-defined expression profile 167. An example of how this type of analysis could be used is to find genes that are expressed at early times in a timecourse, but not at late times. The users set a pattern using the Pull down menus 166 for each condition in a project. The first menu determines whether the user wants genes that are expressed at levels higher than, lower than or equal to 168 the threshold set in the next pull down menu. The threshold is relative to the condition designated as the Control (indicated by [C]) 169. For example setting a condition to “>1.5” would screen for genes that are expressed at levels at least 1.5 times those of the Control. If the user wants all the conditions set to the same direction and threshold the “Set All” menus 164 will achieve this goal rather than setting each condition individually. To begin, the user selects Search 163 and a list of genes with expression profiles matching the set pattern will be displayed. To change the pattern and search again, the user selects the Search pattern button. Pattern navigation 165 uses the Pearson Correlation coefficient to determine whether gene expression patterns match the user-defined pattern. This coefficient can be calculated two ways, centered and un-centered. Generally Un-centered will return more hits, but this can depend on the number of groups in the project. The number to the left of the Centered/Un-Centered pull down menu 161 is the correlation coefficient threshold for this method. The closer the value is to 1, the better the match. The genes listed after searching are sorted by correlation coefficient, so the best matches are always at the top of the list. Using values between 0.95 and 0.99 will insure good matches. Parameters include the Show option 143 which controls how many genes will be displayed on page at one time and the Statistics option 149. Selecting Anova will perform analysis of variance for each gene profile to determine whether there are significant differences in expression for that gene across the project. Significance is determined at p less than 0.05 and is indicated by a blue star to the right of the expression profile.

### FIG. 4F Project Analysis Pattern Summaries 170

FIG. 4F details the expression profiles matching the user-defined pattern 170. Color coding indicates the direction and degree of regulation. Green indicates down regulation relative to the control. Red indicates upregulation. The user can select the Profile 174 or Gene name 177 to view more information about the respective gene. To create a new profile, the user can select the Search Pattern button 175. The user may also select Export Results (not shown) functionality to export the results of this analysis in tab delimited format.

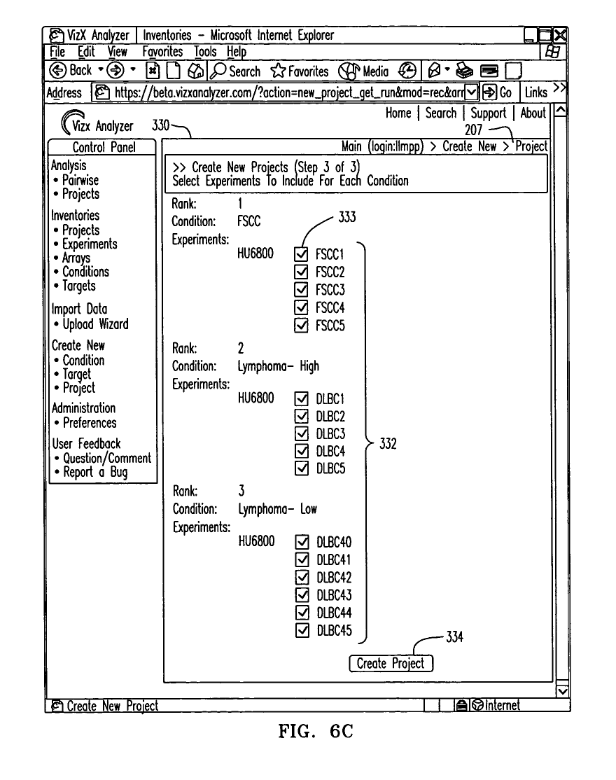

### FIG. 5 User Preferences 180

The User Preferences section 180 contains the features where users can set various parameters for their accounts. System help such as the availability of on-line help can be Turned on or off 182. Display of results returned can be controlled by the Results display pick box 183. Data upload default parameters such as set default array platform for Uploading 184 are selected at this screen. The detail of information displayed is selected by Extended stats for project Gene Summaries 186. Gene titles are controlled by the feature Use UniGene titles rather than array annotation for gene names 188. Finally, user information is specified by the User Information section 189.

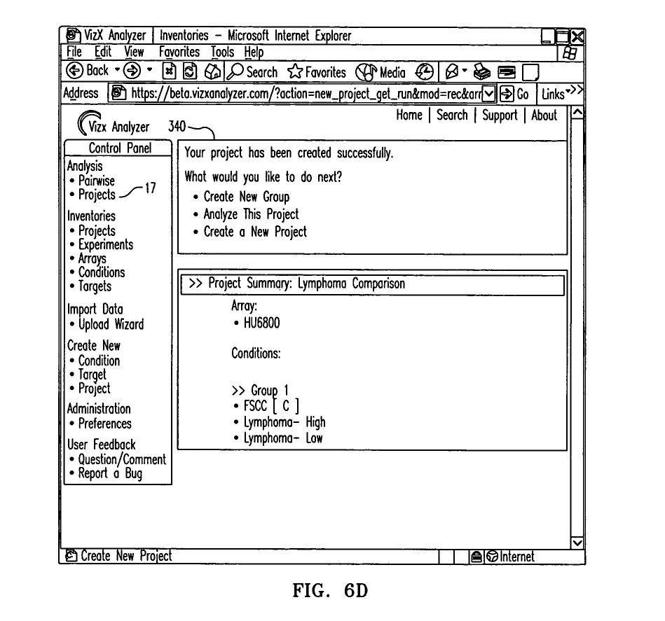

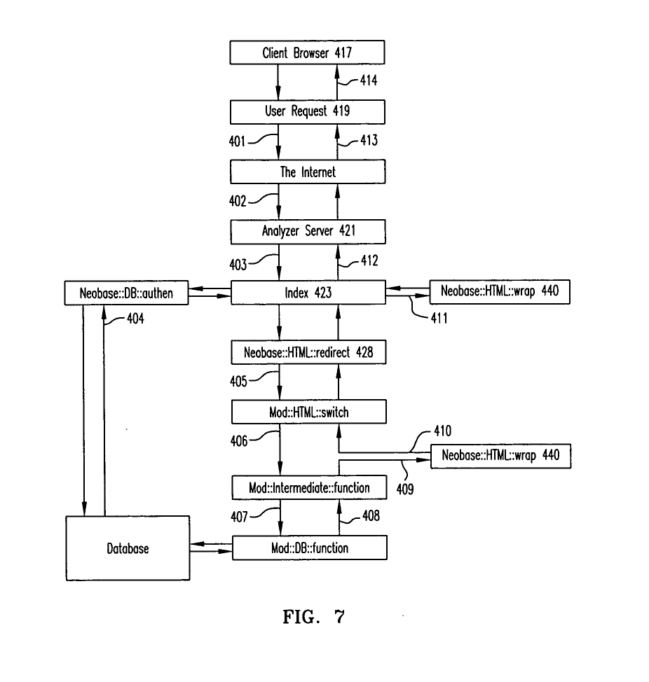

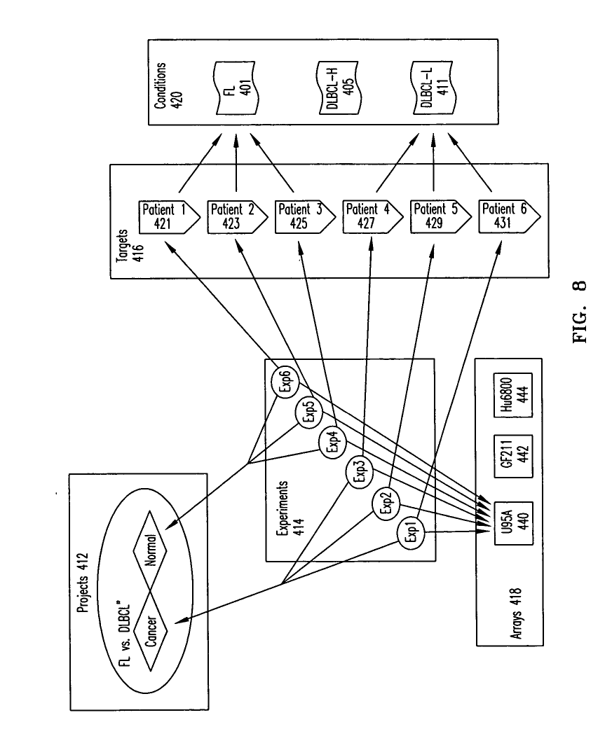

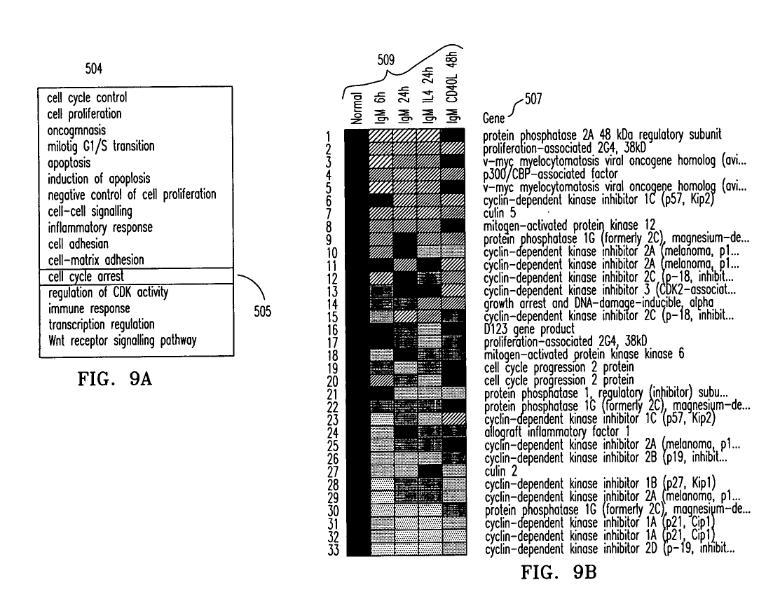

### FIG. 6A Create New Project Array Selection 200

A project 207 is a user-defined set of experiments grouped by experimental condition. Setting up a project allows users to analyze expression across more than two groups. To create a project, the user selects Create New from the section of the Control Panel. The user will see a list 210 of available arrays 211. The user enters a Project Title 203 and Description 205. The user then selects an array 211 or arrays 211 for use in the project 207. As the user selects arrays corresponding lists of experimental conditions 212 that have been examined on that array will be displayed. If more than one array is selected, a list of conditions that a common to all arrays selected will be displayed. To proceed, the user selects Continue 213 after an array or arrays have been selected.

### FIG. 6B Create New Project Condition Selection 215

The user selects conditions to include in Project 207 from list of all conditions available for the selected arrays 212. The user can then select a Normalization method 217 for each array to be included in Project 207. This is followed by selection of conditions 219 from the Available conditions box 225 on the left to include in the project. The user then clicks on the condition to be included in the project. The user clicks the > button 227 to move it to the Selected Conditions box 220 and continues until all of the desired conditions are included in the group. Once conditions have been moved, select conditions and use the Up 222 and Down 224 buttons to reorder them if needed. Conditions can be removed by using the < button 229. Select Create group 226 after conditions have been selected and ordered. Please note, the order of the conditions in the list will determine how the conditions are displayed when projects are analyzed. The first condition in the list will be treated as the control value 225, resulting in the expression values for other members of the project to be expressed relative to this conditions value.

### FIG. 6C Create New Project Experiment Selection 330

Following Condition Selection, the user then selects individual experiments 332 to include in each experimental condition. To select the individual experiments to include for each Condition listed, the user clicks the check box 333 to include a particular experiment. To conclude, the user selects Create Project 334 when all experiments have been selected. The values used for analyzing a project will be the mean of all the experiments selected for that condition.

### FIG. 6D Create New Project Project Created 340

Once complete, the project can then be analyzed. The user can now add another group to a completed project, analyze that project or create a new project by selecting the appropriate link from the list of choices.

To analyze a project, the user selects Projects 17 from the Analysis menu in the Control Panel. This is followed by selection of the magnifying glass shaped Analyze icon (not shown) for the project to be analyzed from the list of available projects. If no projects are available, the user can then create a project. Once a project is selected, a new window will open with analysis options for that project. There are two general type of analysis available, Gene Navigation and Pattern Navigation.

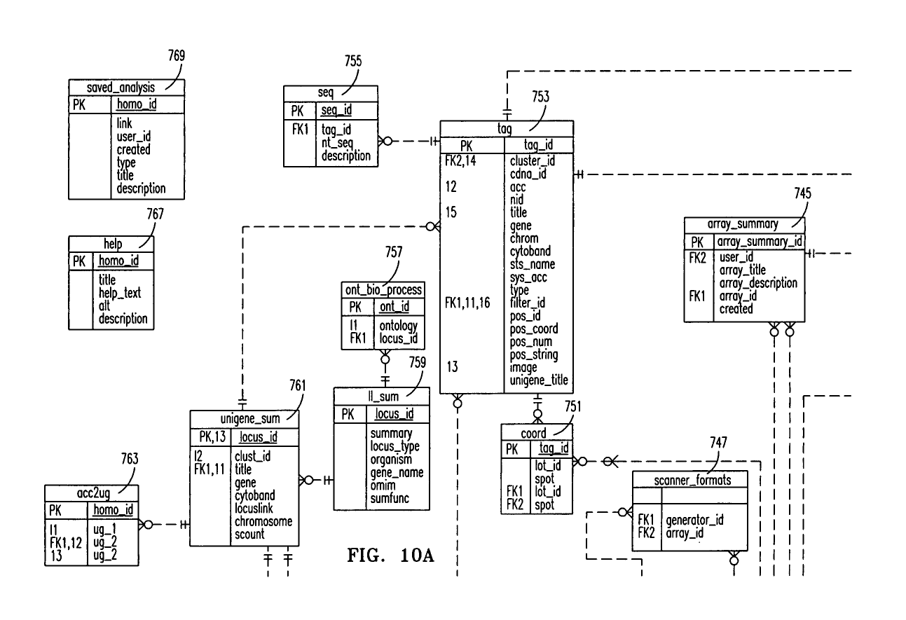

### FIG. 7 Analyzer System Description

Analyzer uses a combination of Perl, a web server and a relational database to process and display the results of user requests for analysis. The client is a standard browser. Presented with what is essentially a web page, the user uses links and buttons to request analysis 401. The request is sent in encrypted form via the internet to an analyzer server using standard HTTP protocols 402. The analyzer server receives the request 403 via the web browser which is then passed to the authentication means. The user is authenticated 404 against the database and, once authenticated, the request is passed to the main switching algorithm 405. The switching algorithm determines what general area the user's request needs to be directed to, i.e., data analysis, data upload, record management, etc. The request is then sent to a secondary switching algorithm 406 which determines the appropriate function calls to process the request. Typically, this involves a database call to get the needed data 407, the data is returned 408 and some processing and analysis 409 takes place. After the data has been analyzed, it is passed to a formatting function that creates a report in HTML or PDF format 410. The report is then passed back to each switch. Some final formatting is performed 411 before the report is returned to the web server which encrypts it 412. At this point the encrypted report 413 is sent back to the user via the internet where the browser decrypts and renders the report 414.

Walk through of how the method and related device operates using Pairwise Comparison:

Using the browser 417 such as Internet Explorer or Netscape, the user would select the Experiments that are to be compared using the checkboxes, select the various parameters for the comparison then hit the ‘Analyze’ button. Browser 417 then encrypts and sends the request 419 to the Analyzer server 421 where the user is authenticated. Index.pl 423 receives the authenticated user and the request using CGI. The request is then passed to Neobase::HTML::redirect 428 which examines the request and determines that, in this case, it needs to passed to the Array module since this is a request for analysis. It is therefore passed to Array:HTML::switch (not shown) which further examines the request. Array::HTML::switch (not shown) determines that this is a request for pairwise so the request is sent to the appropriate function to begin the pairwise analysis—Array::Compare::pairwise (not shown). This function takes information in the request to determine which Experiments are being compared and uses Array::Data::New (not shown) which in turn uses Array::DB::get_run_data (not shown) to retrieve the data from the database for each Experiment and build the data structures. The data is then returned to Array::Compare::pairwise (not shown). This function further uses statistical functions Array::Stats::average and Array::Stats::compare to apply statistical methods (not shown) to the data. The results of the analysis are sent to Array::HTML::pairwise_results (not shown) where a report for this specific analysis type is created. Once the report is created, it is sent back through the switching algorithms to Neobase::HTML::wrap 440 where final formatting is performed. The report is then sent back to server 421, where it is encrypted and sent back to the user. The user's browser 417 decrypts and renders the report displaying the results (not shown).

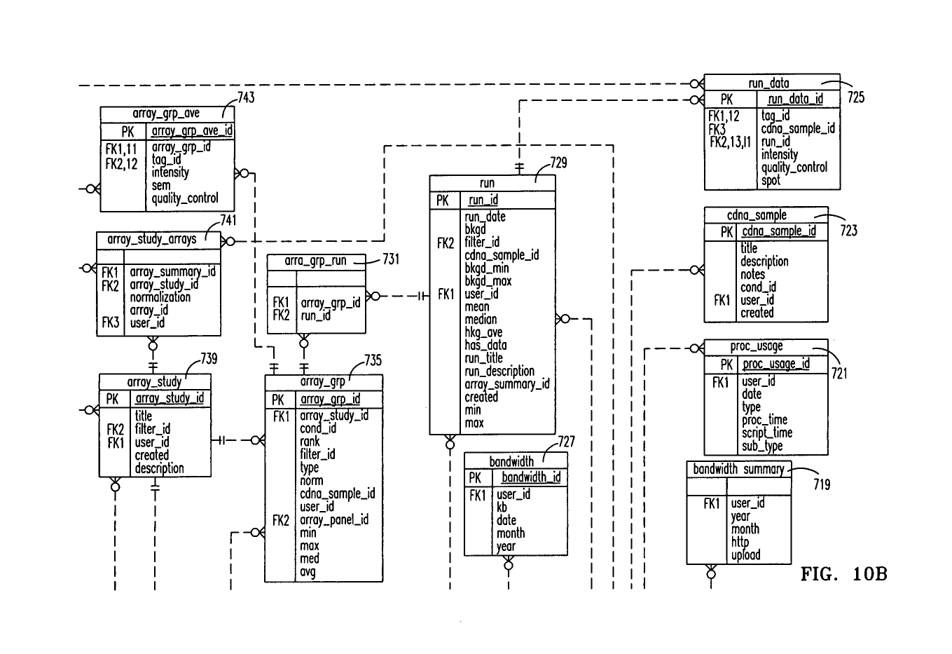

### FIG. 8 System Schematic

FIG. 8 is a schematic showing how data is organized and giving examples of the types of relationships that exist. The schematic of FIG. 8 is also intended to provide a framework for a representative Pairwise Comparison of experiments 414 detailed in the tables below. In FIG. 8, a selection of microarrays 418 from two different vendors are exposed to a biological sample (not shown). Experiments 414 are the result of the combination of a Target 416 and a data source such as Arrays 418. Targets 416 refer to individual cDNA/mRNA samples. In the scenario depicted in FIG. 8, the user might take a cDNA sample from each patient (not shown). A cDNA sample from one patient would be of the condition FL 401 and a sample from another patient would be of the condition DLBCL-H 405 or DLBCL-L 411. With more particularity, the user or assistant to the user exposes cDNA 416 to arrays 418 and receives a set of results. For example, cDNA from patient 5 429 (condition DLBCL-L) is exposed to Array U95A 440. Conditions can be thought of as general groupings. For example, in a cancer study a user might have one set of cancer patients with particular treatment characteristics and one set of patients with cancer that did not exhibit those characteristics. In the working example presented in FIG. 8, all patients may have had a particular type of cancer but have had different genes expressed as a consequence of the treatment.

Experiments 414 are a collection of array hybridization events (An array, a target and the data associated with that hybridization. The example compares Follicular Lymphoma 401 against Diffuse Large B Cell Lymphoma 405 and 411. The example also compares 2 groups of DLBCL patients 421, 423, 425, 427, 429, 431. One group (DLBCL-High) had a very high survival rate following treatment, the other (DLBC-Low) had a very low survival rate. The goal of the example is to show how Pairwise Comparison can assist in finding genes that can distinguish FL 401 from both types of DLBCL 405, 411. The Experimental Conditions (or other group designation) associated with a target in this case are either Follicular Lymphoma 401 or Diffuse Large B Cell Lymphoma-High 406 or Diffuse Large B Cell Lymphoma-Low 411, but could also be a time point, a treatment, tissue type or cancer type. The example also serves to identify genes that are up regulated only in the DLBCL-Low 405 group. Targets in this example refer to the cDNA (or RNA) sample which is labeled and put onto the respective slide or chip. There are six different 421, 423, 425, 427, 429, 431 targets 416 representing B cell samples (not shown) from 6 individuals grouped by 3 conditions 401, 405, 411.

To identify genes that distinguish FL 401 from DLBCL 405, 411 the user can perform a pairwise comparison with the FL results in Group 1 and all the DLBCL results in Group 2. A project 412 containing all 3 conditions 420 can be created (with the FLs as a control) and then Pattern Navigation can be used to find genes upregulated in the DLBCL-Low group. Using the Gene Ontologies™ functionality, the user can also use gene navigation to examine the expression of Apoptosis genes as a predictor that these genes could affect how well the B cells respond to treatment.

In contrast to presently available methodologies, the system and device provides several features that allow users to overcome present difficulties and easily compare expression data from different platforms. Comparison of expression data is termed Pairwise Comparison. Data can be accepted in multiple array formats 418; users can load data from both Affymetrix GeneChips and cDNA spotted arrays. The disclosed method and related device can automatically convert gene annotation provided by array manufacturers into the most current UniGene annotation, ensuring that the same genes will always have the same title according to the method regardless of what information the manufacturer originally provided to the user. The method and related device can also determine whether two different Accession Ids and/or Image Clone IDs represent the same gene.

An example of the use of these features is provided by a comparison of data from Shipp et al (Nature Medicine, Volume 8, Number 1, January 2002) comparing gene expression in Follicular Lymphoma (FL) versus Diffuse Large B Cell Lymphoma (DLBC) using the Affymetrix HU6800 GeneChip 444 with data comparing the same two lymphomas published by Alizadeh et al. (Nature 403:503-511, 2000) using a spotted cDNA arrays (Lymphochip) 440, 442. Data from both groups can be loaded into the present method and related device, a Project 412 can then be created using all arrays and including both FL and DLBCL as conditions. Using Gene Navigation, particular genes could be selected and the expression of genes on both arrays can then be calculated for DLBCL relative to FL. Sorting the genes alphabetically and using UniGene titles would list the same genes next to each other regardless of annotation provided by the array manufacturer and regardless of whether the accession id used to represent that gene was the same on both platforms. These features would allow users to compare expression of particular genes in the two studies or to compare these two published studies to their own examination of Follicular and Diffuse Large B Cell Lymphomas regardless of the arrays used.

Table 2 represents the underlying data comparing Breast Cancer cells against Normal Cells. Based upon samples from 6 different individuals (6 patients with a variety of conditions), 6 different targets can be labeled for example,

| Target | Condition |
|---|---|
| Patient 1 FL-4 | FL |
| Patient 2 FL-9 | FL |
| Patient 3 DLBCL-1 | DLBCL-High |
| Patient 4 DLBCL-12 | DLBCL-High |
| Patient 5 DLBCL-42 | DLBCL-Low |
| Patient 6 DLBCL-51 | DLBCL-Low |

The user could perform six experiments by hybridizing the six targets on a GeneChip™. The six experiments could then be grouped by condition and analyzed yielding three groups (FL, DLBCL-High and DLBCL-Low) with three sets of data for each group.

Table 3 depicts the expression of Cyclin D1. The lower number indicates lower expression in FL. Cyclin D1 expression is lower in FL than DLBCL in both sets of experiments. Cyclin D1 is represented twice on the Lymphochip (L) and once on HU6800 (H). While numerical data representation is presented here, it is an intended variant that the differences could be presented to the user graphically based upon changes in coloration instead of numerically.

TABLE 3

| | DLBC | FL | Gene |
|---|---|---|---|
| 1 | 3 | 2 | Cyclin D1 (PRAD1: parathyroid adenomatosis 1) L |
| 2 | 3 | 1 | Cyclin D1 (PRAD1: parathyroid adenomatosis 1) H |
| 3 | 3 | 2 | Cyclin D1 (PRAD1: parathyroid adenomatosis 1) L |

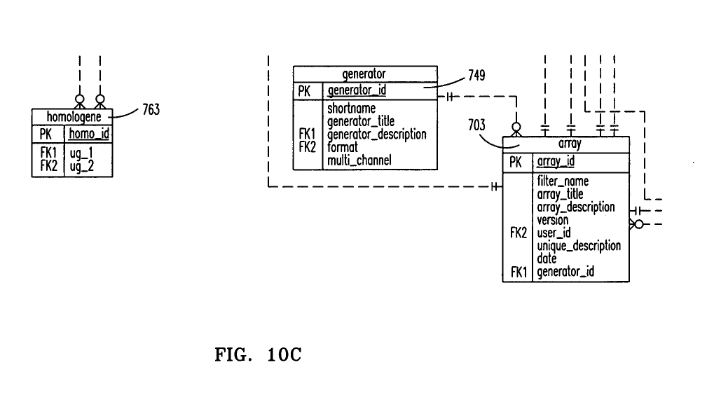

### FIG. 9

The previous examples are by no means intended to be limiting or representative of the scope of the various embodiments. FIG. 9 depicts a more comprehensive application of the Gene Ontologies functionality in viewing results according to biological functionality.

The method and related device provides several features that allow users to view expression profiles of groups of genes selected based on their biological function. UniGene and LocusLink summary information can be provided for each gene on an array. Gene Ontology™ designations from LocusLink are integrated into this annotation. As new ontology designations are added to LocusLink, this information is automatically added to the annotation for a user's genes. Users can than search for groups of genes on their arrays using this information.

The “Search by Gene Function” method for Project analysis provides a list of available Gene Ontolgies™. An ontology of interest can be selected and a search performed. All genes on the arrays included in that project and having that ontology designation as part of their annotation are selected and an expression profile for each of the genes is created. Gene sets can then be sorted based on expression profile and statistical analysis can be applied to these datasets. These features allow users to view their expression data in the context of biological processes.

A small but by no means comprehensive list of Biological processes 504 are listed on the left, with corresponding expression profiles for the selected Cell cycle arrest 505 are detailed on the right. Expression profiles 509 for Cell Cycle Arrest genes 507 created using the method and related device Search by Gene Function feature. While results are graphically represented, they could just as easily be numerically represented as well.

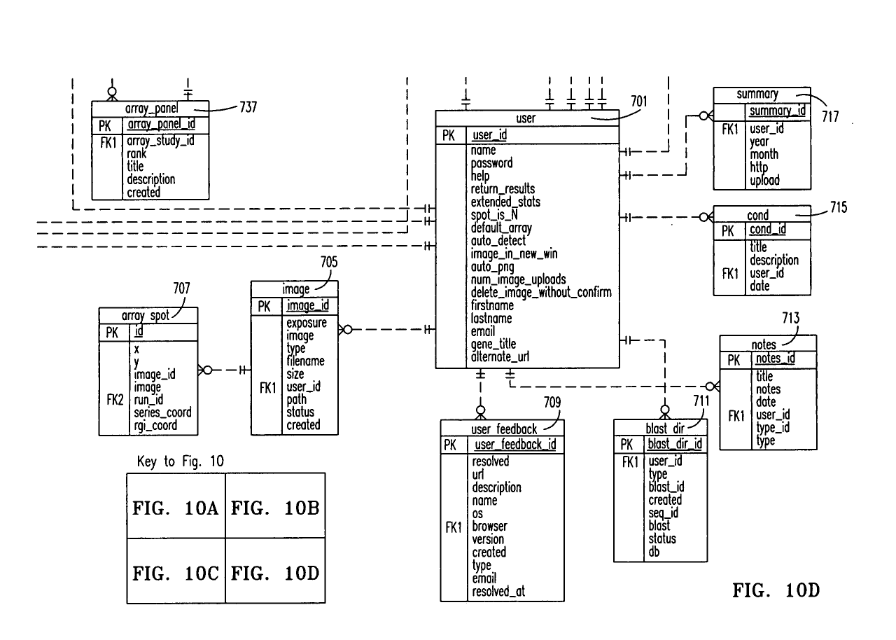

### FIG. 10 Database

FIG. 10 is a relational database structure according to the present method and related device. User table 701 contains fields for information about the user including login info and preferences. Array table 703 contains fields for manufacturer information about each microarray in the database. Image table 705 contains fields for information about upload images. Array_spot table 707 contains fields for information about each spot in an uploaded image. User_feedback table 709 contains fields for user comments about the system. Blast_dir table 711 contains fields for blast requests submitted by users. Notes table 713 contains fields for notes submitted by users about their various records. Cond table 715 contains fields for condition information. Summary table 717 contains fields for future use for summary information. Bandwidth_summary table 719 contains fields for bandwidth usage for each user. Proc_usage table 721 contains fields for computer processor usage for each user. Cdna_sample table 723 contains fields for target/cdna information. Run_data table 725 contains fields for intensities and qualites for each experiment. Bandwidth table 727 contains fields for bandwidth usage for each user. Run table 729 contains fields for experiment information. Array_grp_run table 731 contains fields for which experiments are in a project group. Array_grp table 735 contains fields for each group in a project. Array_panel table 737 contains fields for each array in a project. Array_study table 739 contains fields for project information. Array_study_arrays table 741 contains fields for each array in a project. Array_grp_ave table 743 contains fields for the average of each group. Array_summary table 745 contains fields for user information about each array for which they have uploaded data. Scanner_formats table 747 contains fields for which scanners (3rd party image processing software) read which arrays. Generator table 749 contains fields for which arrays belong to which scanners. Coord table 751 contains fields for the physical location of a spot on an array. Tag table 753 contains fields for information about the genes at each spot on an array. Seq table 755 contains fields for gene sequences. Ont_bio_process table 757 contains fields for biological process Ontologies. Il_sum table 759 contains fields for locus link summary information. Unigene_sum table 761 contains fields for unigene summary information. Homologene table 763 contains fields for homologene information. Acc2ug table 765 contains fields for accession number to unigene id relationships. Help table 767 contains fields for online help documentation. Saved_analysis table 769 contains fields for saving an analysis process so that it can be repeated at a later time.

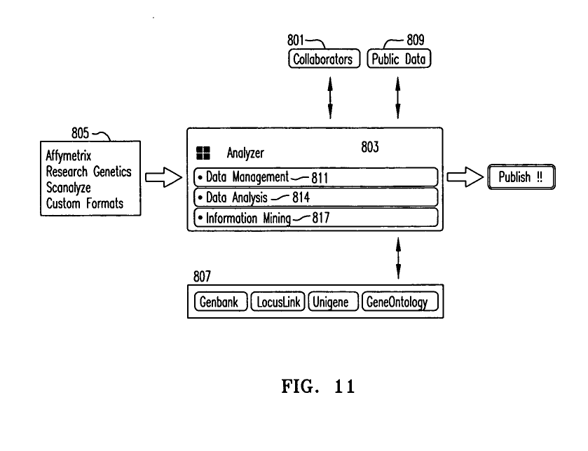

### FIG. 11 Overview

FIG. 11 is an overview of the various elements which make up the method and related device. Within the biological information central server 803, remote users 801 can collaboratively access and share biological information 805. Biological information 805 can be managed 811, undergo mathematical and graphical data analysis 814 as well as information mining 817. In addition, the method and related central server device 803 joins remote users 801 with a central information repository 803 to relate biological information 805 to other datasets such as public data 809 as well as internal functionality and various internet-based public and private human genome registries 807.

### INDUSTRIAL APPLICABILITY

The disclosed method and related device has industrial applicability in the life sciences and biomedical arts. The disclosed method and related device provide enhanced bioinformatics capabilities which allow for remote users to access and interpret their information as well as collaborate with colleagues without restriction on their respective locations.

1. A method for evaluating biological results comprising the steps of:
authenticating one or more remote users with a network server over a network,
selecting biological information to be analyzed by said server,
uploading said biological information to said server,
choosing one or more biological information processing requests,
transmitting biological information processing requests over said network,
processing said biological information according to said biological information processing request, and
returning results to said one or more users over said network.

2. The method of claim 1 wherein said biological information comprises microarray data.

3. The method of claim 2 wherein said processing additionally comprises a filtering step wherein processing of said microarray data includes microarray spot quality control.

4. The method of claim 1 wherein said biological information processing request is submitted by a remote user who did not upload said biological information to said server.

5. The method of claim 1 wherein said processing step additionally comprises an update of gene annotation information.

6. The method of claim 1 wherein said biological information uploading takes place over a secure network.

7. The method of claim 1 wherein said biological information comprises microarray data in the format selected from the group consisting of Affymetrics, Pathways or Scanalyze data formats.

8. The method of claim 7 wherein said biological information additionally comprises cDNA target information user annotation and raw data.

9. The method of claim 1 wherein said processing step additionally comprises clustering said biological information by corresponding biological function.

10. The method of claim 1 wherein said information processing request includes a search step wherein said processing includes a gene sorting step selected from the group consisting of accession ID, image clone ID or cluster ID.

11. The method of claim 1 wherein said processing step additionally comprises a pattern navigation step wherein said users can search for genes matching user defined expression patterns.

12. The method of claim 1 wherein said results additionally comprise expression profiles of groups of genes based upon their biological function.

13. The method of claim 12 wherein said biological function is chosen from the group consisting of cell cycle control, cell proliferation, oncogenesis, mitotic G1/S transition, apoptosis, introduction of apoptosis, anti-apoptosis, negative control of cell proliferation, positive control of cell proliferation, cell-cell signaling, intracellular signaling cascade, inflammatory response, cell adhesion, cell-matrix adhesion, cell cycle arrest, regulation of CDK activity, immune response, transcription regulation and wnt receptor signaling pathway.

14. The method of claim 1 wherein said authenticating step is computer independent, providing said users access from any Internet connected device.

15. A device for evaluating biological results comprising one or more Internet enabled remote user devices, connected to a server for authenticating said one or more Internet enabled remote users devices over a network,
said one or more Internet enabled remote user devices containing an Internet connection for transmission of biological information and user biological information processing requests,
said server containing a relational database for receiving biological information to be analyzed by said server,
said server incorporating a processor for processing said biological information according to said user requests,
said server additionally incorporating an Internet connection for receiving returning results to said one or more users.

16. The device of claim 15 wherein said processor means returns said results based upon a single action by said one or more users.

17. The device of claim 15 wherein said processing means processes said biological information into said results according to the clustering of biological results by biological function.

18. A method of evaluating biological results comprising:
under control of a client system,
displaying information identifying the biological information; and in response to only a single action being performed, sending a request to analyze the biological results along with an identifier of the user to a server system;
under control of a single-action analysis component of the server system, receiving the request;
retrieving additional biological information previously stored for the user identified by the identifier in the received request; and
generating biological results for the user identified by the identifier in the received request using the retrieved additional information; and
returning to the user the requested biological results based upon the previously uploaded biological information.
